(19) United States
(12) Patent Application Publication
(10) Pub. No.: US 2017/0217861 A1
(43) Pub. Date: Aug. 3, 2017

(54) Method and Apparatus for Separation of Colloidal Suspension from a Solution of Organic Compound, such as Monoethylene Glycol

(71) Applicant: Reliance Industries Limited, Mumbai (IN)

(72) Inventors: Nemani Venkateswara Rao, Kakinada (IN); Mandava Venkata Subbarao, Vijayawada (IN); Adda Rama Rao, Kakinada (IN); Akshya Kumar Bakshi, Kakinada (IN); Maddirala Krishna, Kakinada (IN); Anandan Arumugam, Chennai (IN)

(21) Appl. No.: 15/328,231

(22) PCT Filed: Jul. 23, 2015
(86) PCT No.: PCT/IB15/55578

§ 371 Date: Jan. 23, 2017

(30) Foreign Application Priority Data
Jul. 24, 2014 (IN) 2034/MUM/2014

(51) Int. Cl. C07C 29/86; B01D 21/01; B01D 53/14; C07C 29/76

(52) U.S. Cl. CPC C07C 29/86; C07C 29/76; B01D 21/01; B01D 53/1425; B01D 2252/2023

(57) ABSTRACT

The present disclosure relates to a method for separation of colloidal suspension from a solution of organic compound, wherein the solution of organic compound includes but is not limited to hydrocarbon based solution. More particularly the method provides for removal of colloidal suspension from the solution to obtain clarified hydrocarbon based solution including but not limiting to the compound(s) from the diol family such as mono ethylene glycol (MEG), wherein said removal is achieved by contacting the solution with at least one flocculant followed by chemicals including but not limiting to precipitants. The disclosure also provides a system for carrying out the method of separating colloidal suspension from a solution of organic compound.

### TECHNICAL FIELD

The present disclosure relates to a method for separation of colloidal suspension from a solution of organic compound, wherein the solution of organic compound includes but is not limited to hydrocarbon based solution. More particularly the method provides for removal of colloidal suspension from the solution to obtain clarified hydrocarbon based solution including but not limiting to the compound(s) from the diol family such as mono ethylene glycol (MEG), wherein said removal is achieved by contacting the solution of organic compound with at least one flocculant followed by chemicals including but not limiting to precipitants. The present disclosure also provides a system for carrying out the method of separating colloidal suspension from a solution of organic compound including but not limiting to hydrocarbon based solution.

### BACKGROUND AND PRIOR ART OF THE DISCLOSURE

Hydrate inhibitors such as mono-ethylene glycol (MEG) are used in hydrocarbon gas and/or condensate pipelines to absorb moisture and prevent hydrate forming in the pipe, which can lead to blockage and corrosion. Typically, the MEG (or other inhibitor) is injected into the upstream end of the pipeline, and is separated from the hydrocarbon fluids at a receiving facility at the downstream end. The separated MEG (known as rich MEG) which carries absorbed water is regenerated by a water removal process to produce “lean MEG” for re-use. However, hydrate inhibitors such as MEG also tend to become polluted by other components in the pipeline. Some of the pollutants, for example, pipeline corrosion products and scale are present as particles in the flow stream. Others, such as hydrocarbons, salts from formation water or production chemicals are present in solution, but may precipitate as small particles during the MEG regeneration process.

Removal of these (pollutants and hydrocarbons) particles is important for the performance of the MEG regeneration process, because the particles tend to accumulate in the regeneration process, and clog process equipment. One known solution to this problem is to separate the particles by introducing a solid separation unit and a desalination unit (reclaimer) into the process. A problem with these approaches is that significant quantities of the particles are very fine and difficult to separate. Separation processes and equipment for handling these particles are large, expensive, operator intensive and prone to failure due to clogging. In some applications MEG reclamation may be carried out on off-shore platforms where space is at a premium.

Also, depending on operating conditions, which may change over the operational life of the plant, the distribution of particle size may vary significantly. This makes it difficult to design a system that can perform reliably for all operating conditions over the life of the plant.

In order to overcome the limitations of the conventional processes for separating colloidal suspension from hydrocarbon based solution such as rich MEG in the flow lines, the present disclosure provides an innovative method, wherein the impurities including but not limiting to salts, which tend to scale/foul downstream equipments and pipings are removed efficiently and a clarified MEG is obtained.

### SUMMARY OF THE DISCLOSURE

The present disclosure relates to a method for separation of colloidal suspension from a solution of organic compound(s), said method comprising steps of: (a) contacting the solution of organic compound with at least one flocculant, followed by incubating the solution; and (b) contacting the solution of step (a) with at least one precipitant and allowing the solution to settle for separation of the colloidal suspension from the solution of organic compound.

The present disclosure further relates to a system for separation of colloidal suspension from a solution of organic compound, the system comprising: (a) a first flash drum adapted to receive mixture of solution of organic compound and first flocculant, and is configured to incubate the mixture; and (b) a second flash drum fluidly contacted to the first flash drum, the second flash drum is adapted to receive the solution of organic compound from the first flash drum with a second flocculant, and is configured to incubate a mixture of the solution of organic compound and the second flocculant; and (c) a settling tank fluidly contacted to the second flash drum, the settling tank is adapted to receive the solution of organic compound from the second flash drum with precipitant for the separation of colloidal suspension from the solution of organic compound.

BRIEF DESCRIPTION OF ACCOMPANYING FIGURES

In order that the disclosure may be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present disclosure where:

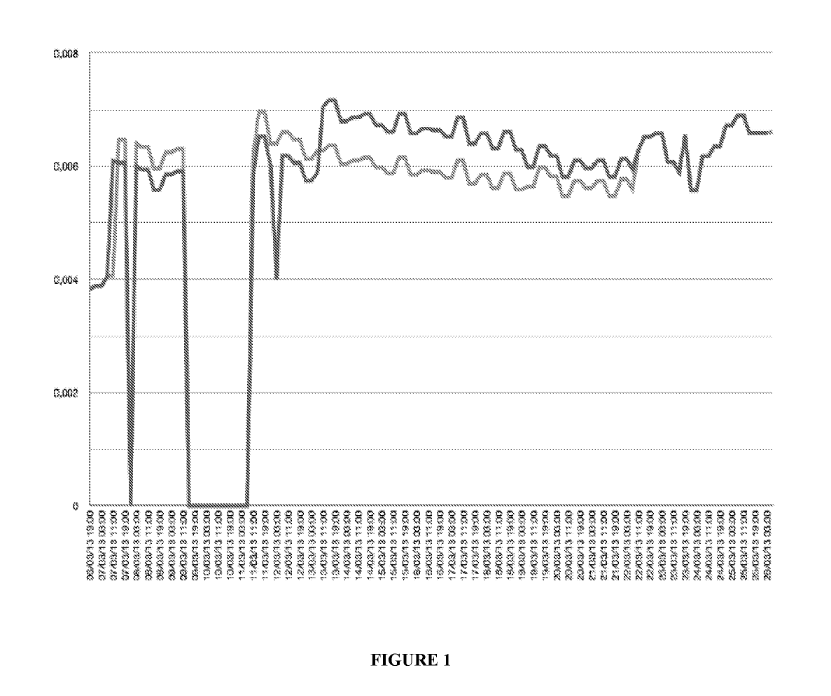

FIG. 1 illustrates the percentage dosing of first flocculant and second flocculant.

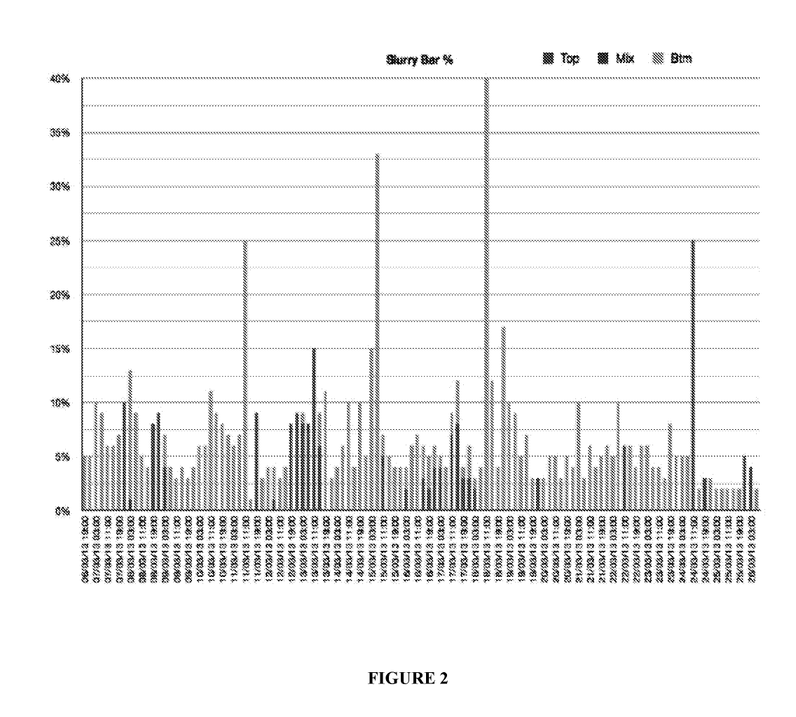

FIG. 2 illustrates the percentage of slurry formation and location (top or middle or bottom) of the slurry in the settling tank.

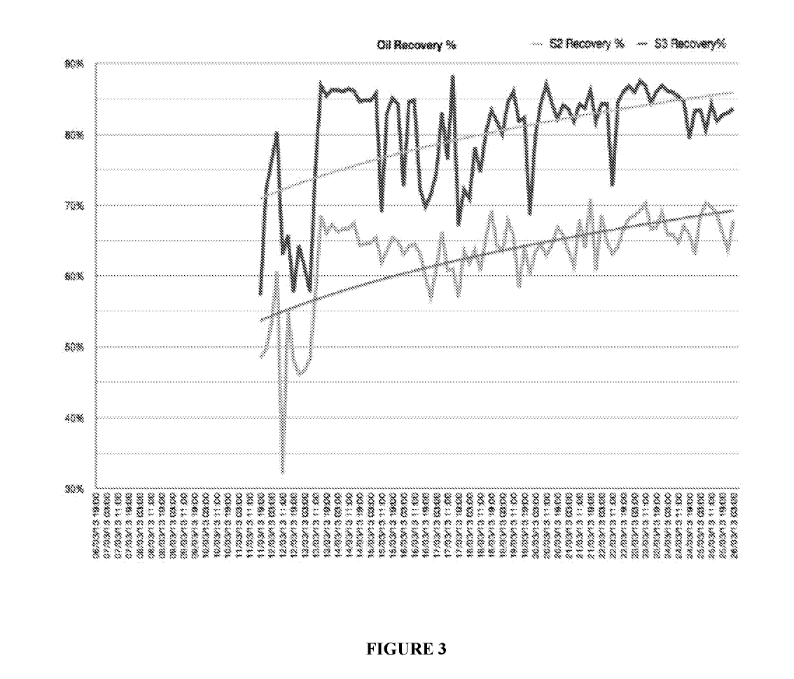

FIG. 3 illustrates the percentage oil recovery.

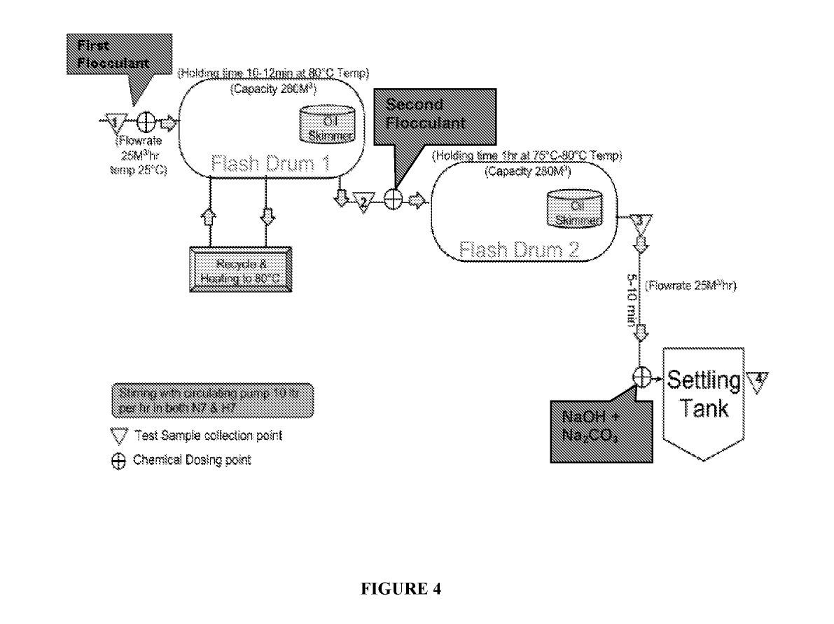

FIG. 4 illustrates the schematic representation of the system employed in the present disclosure.

### DETAILED DESCRIPTION OF THE DISCLOSURE

The present disclosure provides a method for separating colloidal suspension from a solution of organic compound, by contacting the solution with at least one flocculant followed by chemicals including but not limiting to precipitants. The solution of organic compound subjected to said separation includes but is not limited to organic compound comprising hydrocarbon, organic compound comprising oxygen and organic compound comprising nitrogen, preferably organic compound comprising hydrocarbon such as mono ethylene glycol (rich MEG).

The term ‘colloidal suspension’ used within the purview of the instant invention refers to a mixture wherein particles of a substance are present within a fluid but remain undissolved.

The term ‘colloidal suspension’ used within the purview of the instant invention refers to a mixture with properties of either a solution or fine suspension or combination thereof. The term ‘colloidal suspension’ used within the purview of the instant invention refers to a solution in which a material is evenly suspended in a liquid. The term ‘colloidal suspension’ used within the purview of the instant invention refers to a mixture of usually two materials where one is dispersed in the other at a microscopic level, hut not chemically bonded to it. The ‘colloidal suspension’ of the present disclosure intends to capture any liquid solution having either another liquid or any soluble or insoluble suspended particles therein, or any combination thereof.

In an exemplary embodiment, the present disclosure provides a method for separating colloidal suspension from a solution of organic compound, wherein the solution of organic compound includes but is not limited to hydrocarbon based solution such as rich MEG, and wherein the said separation involves contacting the solution with at least one flocculant followed by chemicals including but not limiting to precipitants such as alkali metal hydroxide and carbonate or combinations thereof. In an embodiment, the hydrocarbon based solution further includes but is not limited to petroleum products and natural gas. During the above said separation, the flocculant and chemicals are provided in single or multiple dosages.

In a non-limiting embodiment, the separation of colloidal suspension from the solution of organic compound including but not limiting to hydrocarbon based solution, such as rich MEG, involves contacting the solution with first flocculant followed by second flocculant, within a predetermined time period, followed by contacting the solution with chemicals including but not limiting to precipitants, such as alkali metal hydroxide and carbonate, or a combination thereof. The alkali metal hydroxide includes but is not limited to sodium hydroxide, and the carbonate includes but is not limited to sodium carbonate and potassium carbonate, or a combination thereof.

In another non-limiting embodiment, the separation of colloidal suspension from the solution of organic compound including but not limiting to hydrocarbon based solution, such as rich MEG involves contacting the solution with only one flocculant for a predetermined time period, followed by contacting the solution with chemicals including but not limiting to precipitants, such as alkali metal hydroxide and carbonate, or a combination thereof.

In yet another non-limiting embodiment, the separation of colloidal suspension from the solution of organic compound including but not limiting to hydrocarbon based solution, such as rich MEG involves contacting the solution with first flocculant for a predetermined time period followed by contacting the solution with chemicals including but not limiting to precipitants such as alkali metal hydroxide and carbonate for a predetermined time period and, followed by contacting the solution with second flocculant for a predetermined time period.

In a non-limiting embodiment, the flocculants employed in the method of the present disclosure for separation of colloidal suspension from the solution of organic compound including but not limiting to hydrocarbon based solution such as rich MEG, are esters comprising components including but not limiting to palm oil and kernel oil, each individually at a concentration ranging from about 30% v/v to about 70% v/v. The flocculant optionally comprises water, maximally upto 40% v/v.

In a non-limiting embodiment, the first flocculant comprises components including but not limiting to palm oil, palm kernel oil and water and the second flocculant comprises components including but not limiting to palm oil and palm kernel oil.

In yet another non-limiting embodiment, the first flocculant employed in the method of the present disclosure for separation of colloidal suspension from the rich MEG, comprises palm oil at a concentration ranging from about 40% v/v to about 60% v/v, palm kernel oil at a concentration ranging from about 30% v/v to about 50% v/v and water at a concentration ranging from about 10% v/v to about 40% v/v.

In another non-limiting embodiment, the second flocculant employed in the method of the present disclosure for separation of colloidal suspension from the rich MEG, comprises palm oil and kernel oil, each individually at a concentration ranging from about 30% v/v to about 70% v/v. The second flocculant is devoid of water.

In still another non-limiting embodiment, the first flocculant comprises about 50% v/v of palm oil, about 40% v/v of palm kernel oil and about 10% v/v of water, whereas the second flocculant comprises about 40% v/v to about 60% v/v of palm oil and about 40% v/v to about 60% v/v palm kernel oil.

In a non-limiting embodiment, the separation of colloidal suspension from the solution of organic compound including but not limiting to hydrocarbon based solution, such as rich MEG involves contacting the solution with only one flocculant for a predetermined time period, followed by contacting the solution with chemicals including but not limiting to precipitants, such as alkali metal hydroxide and carbonate, or a combination thereof. In such embodiment, wherein only one flocculant is employed in the method of the present disclosure, the flocculant comprises components including but not limiting to palm oil and palm kernel oil. In other words, when only one flocculant is employed, the flocculant is devoid of water and only comprise palm oil and palm kernel oil, which is also defined as the ‘second’ flocculant within the ambit of the present disclosure.

In an exemplary embodiment, the colloidal suspension to be separated from the hydrocarbon based solution such as rich MEG includes but is not limited to water, corrosion products, salts with moderate to low solubility and unwanted impurities, or any combination thereof.

In a non-limiting embodiment, the moderate to low solubility salt to be separated from the hydrocarbon based solution such as rich MEG includes but is not limited to divalent cations of metal including but not limiting to iron, calcium, magnesium, barium and strontium, or any combination thereof.

In an alternate embodiment, the separation of colloidal suspension from the solution of organic compound including but not limiting to hydrocarbon based solution such as MEG involves contacting the solution with at least one flocculant and chemicals including but not limiting to precipitants at any concentration or ratio thereof.

In an embodiment, the flocculants during separation of colloidal suspension from the solution of organic compound including but not limiting to hydrocarbon based solution such as MEG, leads to flocculation and sedimentation of colloidal suspension including but not limiting to water, corrosion products, salts with moderate to low solubility and unwanted impurities or any combination thereof. In an alternate embodiment, the colloidal suspension to be separated from hydrocarbon based solution such as rich MEG, is referred to as slurry.

In an embodiment, the alkali metal hydroxide employed in the method of the present disclosure as a precipitant maintains the rich MEG solution at an alkaline pH ranging from about 9.5 to about 12. In a preferred embodiment, the alkali metal hydroxide includes but is not limited to sodium hydroxide.

In an embodiment, carbonate is employed in the method of the present disclosure as another precipitant, wherein the alkaline rich MEG solution is contacted with said precipitant to further precipitate or sediment the colloidal suspension including but not limiting to water, corrosion products, moderate to low solubility salts, preferably divalent cations of metal including but not limiting to iron, calcium, magnesium, barium and strontium and unwanted impurities. In a preferred embodiment, the carbonate includes but is not limited to sodium carbonate.

In a non-limiting embodiment, the rich MEG solution is contacted with single or multiple dosages of sodium hydroxide to maintain the MEG solution at an alkaline pH ranging from about 9.5 to about 12, wherein the sodium hydroxide is at a concentration ranging from about 40% w/v to about 50% w/v.

In a non-limiting embodiment, the rich MEG solution is contacted with a single dosage of sodium carbonate having a concentration ranging from about 10% w/v to about 40% w/v.

In another non-limiting embodiment, the rich MEG solution is contacted with multiple dosages of sodium carbonate having a single dosage concentration ranging from about 10% w/v to about 40% w/v.

In yet another non-limiting embodiment, the rich MEG solution is contacted with sodium carbonate having a concentration of about 36% w/v.

In a preferred embodiment, the rich MEG solution is contacted with sodium hydroxide having a concentration of about 50% w/v and sodium carbonate having a concentration of about 25% w/v.

In an exemplary embodiment, the method for separation of colloidal suspension from the solution of organic compound including but not limiting to hydrocarbon based solution such as rich MEG, comprises steps of:
a. contacting the solution with at least one flocculant followed by incubating the solution for a predetermined duration and at a predetermined temperature; and
b. contacting the incubated solution with chemicals including but not limiting to precipitants such as sodium hydroxide and sodium carbonate, and allowing the solution to settle for a predetermined duration and at a predetermined temperature, for precipitation and the separation of the colloidal suspension.

In an exemplary embodiment, the method for separation of colloidal suspension from the solution of organic compound including but not limiting to hydrocarbon based solution such as rich MEG, comprises steps of:
a. contacting the solution with a first flocculant followed by incubating the solution for a predetermined duration and at a predetermined temperature;
b. contacting the incubated solution with a second flocculant followed by re-incubating the solution for a predetermined duration and at a predetermined temperature; and
c. contacting the re-incubated solution with chemicals including but not limiting to precipitants such as sodium hydroxide and sodium carbonate, and allowing the solution to settle for a predetermined duration and at a predetermined temperature, for precipitation and the separation of the colloidal suspension.

In a non-limiting embodiment, in the method of the present disclosure involving contacting the solution to a first and a second flocculant followed by chemicals, the solution is incubated upon addition of the first flocculant for a duration ranging from about 10 minutes to about 15 minutes at a temperature of about 80° C. The solution is re-incubated upon addition of the second flocculant for a time period of about 1 hour at a temperature ranging from about 80° C. to about 90° C.

In a preferred exemplary embodiment, the method for separation of colloidal suspension from the solution of organic compound including but not limiting to hydrocarbon based solution such as rich MEG, comprises steps of:
a. heating the solution at a temperature ranging from about 80° C. to about 90° C. for a duration ranging from about 10 minutes to about 15 minutes;
b. contacting the heated solution with a first flocculant, followed by stirring the solution for a predetermined duration;
c. heating the stirred solution at a temperature ranging from about 80° C. to about 90° C. for a duration ranging from about 10 minutes to about 15 minutes;
d. contacting the heated solution of step (c) with a second flocculant, followed by stirring the solution for a predetermined duration;
e. incubating the heated solution of step (d) at a temperature ranging from about 80° C. to about 90° C. for a predetermined duration, followed by removing any oil layer from surface of the solution;
f contacting the solution of step (e) with sodium hydroxide, followed by stirring the solution for a predetermined duration;
g. contacting the solution of step (f) with sodium carbonate, followed by stirring the solution for a predetermined duration; and
h. incubating the solution of step (g) at a temperature ranging from about 80° C. to about 90° C. for a predetermined duration for allowing the solution to settle, for the separation of colloidal suspension from the solution.

In a non-limiting embodiment, the pH of the solution after addition of sodium hydroxide at a concentration of about 50% is ranging from about 9.5 to about 12. This alkaline pH of the solution leads to better slurry formation, wherein the slurry comprises water, corrosion products, salts with moderate to low solubility and unwanted impurities, or any combination thereof.

In a non-limiting embodiment, the incubation of step (h) in the above mentioned method leads to flocculation and sedimentation of the slurry, wherein the slurry comprises water, corrosion products, salts with moderate to low solubility and unwanted impurities, or any combination thereof.

In an exemplary embodiment, the method for separation of colloidal suspension from the solution of organic compound including but not limiting to hydrocarbon based solution such as rich MEG, is a continuous method, wherein the steps described under the method occur without any interruption and the duration of each step is predetermined to make sure that the flow of the steps within the method occur without any interruption. In a non-limiting embodiment, predetermined duration described in the steps of the method for separation of colloidal suspension from the solution of organic compound refers to a duration of about 1 minute, a duration of about 2 minutes, a duration of about 3 minutes, a duration of about 4 minutes, a duration of about 5 minutes, a duration of about 6 minutes, a duration of about 7 minutes, a duration of about 8 minutes, a duration of about 9 minutes, a duration of about 10 minutes, a duration of about 15 minutes, a duration of about 20 minutes, a duration of about 25 minutes, a duration of about 30 minutes, a duration of about 35 minutes, a duration of about 40 minutes, a duration of about 45 minutes, a duration of about 50 minutes, a duration of about 55 minutes and a duration of about 60 minutes. Thus, the method of the instant disclosure is not limited by the duration described herein, and a person skilled in the art would be aware as to how to perform this method, so that each of the steps remain uninterrupted and continuous.

In another non-limiting embodiment, the steps under the method for separation of colloidal suspension from the solution of organic compound including but not limiting to hydrocarbon solution such as rich MEG occur at a temperature ranging from about 80° C. to about 90° C.

In an alternate embodiment, the method for separation of colloidal suspension from the solution of organic compound including but not limiting to hydrocarbon based solution such as rich MEG, comprises steps of:
a. contacting the solution with a flocculant followed by incubating the solution for a predetermined duration and at a predetermined temperature; and
b. contacting the solution with chemicals including but not limiting to precipitants such as sodium hydroxide and sodium carbonate, followed by incubating the solution for a predetermined duration and at a predetermined temperature, for precipitation and the separation of the colloidal suspension.

In a non-limiting embodiment, prior to contacting the solution of organic compound including but not limiting to hydrocarbon based solution such as rich MEG with a flocculant, the solution is heated to a temperature ranging from about 80° C. to about 90° C. for a duration ranging from about 10 minutes to about 15 minutes.

In a non-limiting embodiment, after contacting the solution of organic compound including but not limiting to hydrocarbon based solution such as rich MEG with second flocculant, the solution is subjected to stirring for a predetermined duration, followed by incubating the solution for a duration ranging from about 80° C. to about 90° C. . Post incubation any oil layer on the surface of the solution is removed by a process including but not limiting to skimming.

In a non-limiting embodiment, when the separation of colloidal suspension from the solution of organic compound including but not limiting to hydrocarbon based solution, such as rich MEG involves only one flocculant, the flocculant comprises components including but not limiting to palm oil and palm kernel oil. In other words, when only one flocculant is employed, the flocculant is devoid of water and only comprise palm oil and palm kernel oil, which is also defined as the ‘second’ flocculant within the ambit of the present disclosure.

In a non-limiting embodiment, the pH of the solution of organic compound after addition of the precipitant such as sodium hydroxide ranges from about 10 to about 11.9, wherein this alkaline pH of the solution along with the presence of the single flocculant and other precipitant such as sodium carbonate in the solution aids in precipitation/sedimentation of colloidal suspension including but not limiting to water, corrosion products, salts with moderate to low solubility and unwanted impurities or any combination thereof, thereby leading to the separation of the colloidal suspension from the solution to obtain clarified hydrocarbon solution such as clarified MEG solution.

In another alternate embodiment, the method for separation of colloidal suspension from the solution of organic compound including but not limiting to hydrocarbon based solution such as rich MEG, comprises steps of:
a. contacting the solution with a first flocculant followed by incubating the solution for a predetermined duration and at a predetermined temperature;
b. contacting the incubated solution with chemicals including but not limiting to precipitants such as sodium hydroxide and sodium carbonate, followed by re-incubating the solution for predetermined duration and at a predetermined temperature; and
c. contacting the re-incubated solution with a second flocculant followed by again incubating the solution for a predetermined duration and at a predetermined temperature, for precipitation and the separation of the colloidal suspension.

In a non-limiting embodiment, prior to contacting the solution of organic compound including but not limiting to hydrocarbon based solution such as rich MEG with the first flocculant, the solution is heated to a temperature ranging from about 80° C. to about 90° C. for a duration ranging from about 10 minutes to about 15 minutes.

In a non-limiting embodiment, after contacting the solution of organic compound including but not limiting to hydrocarbon based solution such as rich MEG with first flocculant, the solution is subjected to stirring for a predetermined duration.

In another non-limiting embodiment, prior to contacting the solution of organic compound including but not limiting to hydrocarbon based solution such as rich MEG with the precipitants such as sodium hydroxide and sodium carbonate, the solution is heated to a temperature ranging from about 80° C. to about 90° C. for a time duration ranging from about 10 minutes to about 15 minutes and any oil layer on the surface of the solution is removed by a process including but not limiting to skimming.

In a non-limiting embodiment, after contacting the solution of organic compound including but not limiting to hydrocarbon based solution such as rich MEG with the precipitants, the solution is subjected to stirring for a predetermined duration.

In a non-limiting embodiment, after stirring, the second flocculant is added to the solution, followed by again stirring the solution for a predetermined duration, and heating the solution to a temperature ranging from about 80° C. to about 90° C. for a duration ranging from about 20 minutes to about 30 minutes.

In a non-limiting embodiment, the pH of the solution of organic compound after addition of the precipitant such as sodium hydroxide ranges from about 11 to about 11.5, wherein this alkaline pH of the solution along with the presence of first flocculant, second flocculant and other precipitant such as sodium carbonate in the solution aids in precipitation/sedimentation of colloidal suspension including but not limiting to water, corrosion products, salts with moderate to low solubility and unwanted impurities or any combination thereof, thereby leading to the separation of the colloidal suspension from the solution to obtain clarified hydrocarbon solution such as clarified MEG solution.

In a non-limiting embodiment, the flocculant including but not limiting to the first flocculant or the second flocculant or both are individually at a concentration ranging from about about 0.005% v/v to about 0.010% v/v, respectively in the method for separation of colloidal suspension from hydrocarbon based solution such as rich MEG. In a preferred embodiment the first flocculant and the second flocculant are independently at a concentration ranging from about 0.007% to about 0.008%.

In another non-limiting embodiment, the sodium hydroxide is at a concentration ranging from about 40% to about 50% and the sodium carbonate is at a concentration ranging from about 10%w/v to about 40%w/v in the method for separation of colloidal suspension from hydrocarbon based solution such as rich MEG. In a preferred embodiment, the sodium hydroxide is at a concentration of about 50% and sodium carbonate is at a concentration of about 25% in the method for separation of colloidal suspension from hydrocarbon based solution such as rich MEG.

In an exemplary embodiment, the method of the present disclosure for separation of colloidal suspension from the solution of organic compound including but not limiting to hydrocarbon based solution such as rich MEG, comprises additional process steps including but not limiting to incubating, skimming and settling.

In a non-limiting embodiment, the method for separation of colloidal suspension from the solution of organic compound including but not limiting to hydrocarbon based solution such as rich MEG comprises the process of skimming during incubation upon addition of at least one flocculant, wherein skimming involves removal of hydrocarbon based components including but not limiting to petroleum products such as oil and natural gas.

In another embodiment, skimming process employed in the methods of the present disclosure improves separation of the colloidal suspension including but not limiting to water, corrosion products, salts with moderate to low solubility and unwanted impurities or any combination thereof, wherein the moderate to low solubility salts suspended within the rich MEG are divalent cations of metals including but not limiting to iron, calcium, magnesium, barium and strontium or any combination thereof. An oil layer is observed on the surface of the solution of organic compound including but not limiting to hydrocarbon based solution such a rich MEG that inhibits the flocculation and sedimentation of the colloidal suspension. Therefore, the process of skimming employed after addition of at least one flocculant during the method for separation of the colloidal suspension, removes the free floating oil from the surface of the solution of organic compound aiding in the flocculation and sedimentation of the colloidal suspension or the slurry material, wherein the colloidal suspension mentioned above is also referred to as slurry material.

Within the ambit of the present disclosure, the steps of the methods herein, where duration is not defined, it is to be understood by a person skilled in the art that the step is meant to be a continuous step to ensure that the method is a continuous method occurring without any interruption and the duration of each step is predetermined to make sure that the flow of the steps within the method occur without any interruption. Thus, the method of the instant disclosure is not limited by the duration described herein, and a person skilled in the art would be aware as to how to perform this method, so that each of the steps and thereby the method as a whole remains uninterrupted and continuous.

In a non-limiting embodiment, the methods described herein for separation of colloidal suspension from a solution of organic compound including but not limiting to hydrocarbon based solution such as rich MEG, provides for about 65% to about 85% oil recovery.

In another non-limiting embodiment, the methods described herein for separation of colloidal suspension from a solution of organic compound including but not limiting to hydrocarbon based solution such as rich MEG, provide for of about 95% to about 100% recovery of clarified hydrocarbon based solution such as clarified MEG.

In a non-limiting embodiment, the percentage settling of the slurry/colloidal suspension from the solution of organic compound including but not limiting to hydrocarbon based solution such as rich MEG to obtain clarified hydrocarbon solution such as clarified MEG, by the methods of the present disclosure is about 95% to about 100%, thereby leading to separation of about 95% to about 100% of colloidal suspension from the solution of organic compound including but not limiting to hydrocarbon based solution such as rich MEG to obtain about 95% to about 100% clarified hydrocarbon solution such as clarified MEG. However, in the conventional methods/processes, the settling of the slurry/colloidal suspension from the solution of organic compound is about 50%, thereby clearly showcasing the advantage associated with the methods of the instant disclosure.

In an exemplary embodiment, the conventional methods/processes of separation of colloidal suspension from a solution of organic compound including but not limiting to hydrocarbon based solution such as rich MEG to obtain clarified hydrocarbon based solution such as clarified MEG does not yield the desired result as observed upon employing the methods of the present disclosure. Although the conventional methods/processes employ flocculants such as water based surfactants, the methods/processes employing such flocculants do not lead to separation of colloidal suspension from the solution of organic compound as observed in the methods of the present disclosure. Further, the conventional methods/process leads to oil recovery of about 60%, when compared to oil recovery of 65% to about 85% observed upon employing the methods of the present disclosure. Similarly, the conventional methods/processes lead to clarified hydrocarbon based solution such as clarified MEG recovery ranging from about 80% to about 90%, when compared to about 95% to about 100% clarified the MEG recovered upon employing the methods of the present disclosure.

In a non-limiting embodiment, the methods of the present disclosure lead to separation of colloidal suspension from a solution of organic compound including but not limiting to hydrocarbon based solution, which comprises at least about 2% of the oil layer. In another non-limiting embodiment, the methods of the present disclosure lead to separation of colloidal suspension from a solution of organic compound including but not limiting to hydrocarbon based solution, which comprises about 2% to about 90% oil layer. Accordingly, the methods of the present disclosure lead to about 65% to about 85% oil recovery, during the separation of colloidal suspension from a solution of organic compound including but not limiting to hydrocarbon based solution. Thus, about 65% to about 85% oil is recovered regardless of the initial amount of oil present in the solution of organic compound.

The present disclosure further provides a system for the afore-described methods for separating colloidal suspension from a solution of organic compound including but not limiting to hydrocarbon based solution such as rich MEG.

In another embodiment, the system for carrying out the methods of the present disclosure of separating colloidal suspension from a solution of organic compound including but not limiting to hydrocarbon based solution such as rich MEG collected from slug catcher comprises equipment including but not limiting to flash drum and settling tank.

In an alternate embodiment, the system for separation of colloidal suspension from a solution of organic compound is disclosed. The system comprises a first flash drum which is configured to receive organic compound from a slug catcher, along with first flocculant. The first flash drum is connected to a heating unit, which heats the mixture of organic compound and the first flocculant to a first predetermined temperature and for a first predetermined time. An outlet of the first flash drum is fluidly connected to an inlet of the second flash drum which receives solution of organic compound from the first flash drum with a second flocculant. The second flash drum is also connected to the heating unit, which heats the mixture of solution of organic compound and the second flocculant to a second predetermined temperature and for a second predetermined time. In an embodiment of the disclosure, the heating unit forms a recycle heating loop. The system further comprises a settling tank fluidly connected to the second flash drum. The settling tank is configured to receive the solution of organic compound with a precipitant. The precipitant enables the precipitation of low solubility salts present in the solution of organic compounds, and thereby separates the organic compounds from the colloidal suspensions. In an embodiment of the disclosure, the inlets of the first flash drum, second flash rum and the settling tank comprises at least one provision for receiving at least one of first flocculant, second flocculant, and the precipitant. An outlet of the settling tank is fluidly connected to a storage tank for storing the organic compound which is free from colloidal suspension for further processing and/or use. The first flash drum and the second flash drum, independently function primarily as a 3-phase separators to separate dissolved gases from the liquid phase and any hydrocarbon condensate from hydrocarbon based solution such as rich MEG. Further, the solution is heated by circulating a recycle stream to enable separation of colloidal suspension including but not limiting to water, corrosion products, moderate to low solubility salts and unwanted impurities, or any combination thereof.

In an exemplary embodiment, the system provides for separation of colloidal suspension including but not limiting to water, corrosion products, moderate to low solubility salts and unwanted impurities, wherein the salts of moderate to low solubility are divalent cations of metal such as iron, calcium, magnesium, barium and strontium. The salts are removed by the system by precipitating as carbonates and followed by settling.

In another embodiment, the divalent cations in the hydrocarbon based solution such as rich MEG have an affinity towards lighter phase particles, preferably hydrocarbons including but not limiting to petroleum products such as oils and natural gas. In order to enhance removal of colloidal suspension including but not limiting to moderate to low solubility salts of cations and corrosion products from the hydrocarbon based solution such as rich MEG, said solution is subjected to a continuous process of skimming within the system, which facilitates removal of said hydrocarbons.

In an exemplary embodiment, the system facilitates methods of the present disclosure for separation of colloidal suspension from hydrocarbon based solution such as rich MEG, by contacting the said solution with at least one flocculant followed by chemicals including but not limiting to precipitants such as sodium hydroxide and sodium carbonate. In an alternate embodiment, the hydrocarbon based solution such as rich MEG is contacted with flocculant and chemicals including but not limiting to precipitants such as sodium hydroxide and sodium carbonate in any concentration or ratio thereof.

In an exemplary embodiment, the system facilitates methods of the present disclosure for separation of colloidal suspension from hydrocarbon based solution such as rich MEG, by contacting the solution with first flocculant followed by second flocculant and further followed by chemicals including but not limiting to precipitants such as sodium hydroxide and sodium carbonate.

In an alternate embodiment, the system facilitates methods of the present disclosure for separation of colloidal suspension from hydrocarbon based solution such as rich MEG, by contacting the solution with only one flocculant, followed by contacting the solution with chemicals including but not limiting to precipitants such as sodium hydroxide and sodium carbonate. The flocculant employed as per these embodiments of the present disclosure includes but is not limited to palm oil and palm kernel oil, devoid of water.

In another alternate embodiment, the system facilitates methods of the present disclosure for separation of colloidal suspension from hydrocarbon based solution such as rich MEG, by contacting the solution with first flocculant, followed by chemicals including but not limiting to precipitants such as sodium hydroxide and sodium carbonate, followed by contacting the solution with the second flocculant.

In an exemplary embodiment, the first flocculant includes but is not limited to palm oil, palm kernel oil and water and the second flocculant includes but is not limited to palm oil and palm kernel oil. Within the system of the present disclosure, the hydrocarbon based solution such as rich MEG is contacted with said flocculant(s) at the inlet of flash drum, upstream of the settling tank.

In another exemplary embodiment, the hydrocarbon based solution such as rich MEG is contacted with the chemicals including but not limiting to precipitants such as sodium hydroxide and sodium carbonate at the inlet of settling tank, downstream of the flash drum.

In a preferred embodiment, the system provides for separation of colloidal suspension from a solution of organic compound including but not limiting to hydrocarbon based solution such as rich MEG, in the following manner:
a. contacting the solution collected from the slug catcher with a first flocculant and thereafter pumping the solution to flash drum 1 at a predetermined flow rate followed by incubating the solution for a predetermined duration and at a predetermined temperature, along with continuous or intermittent skimming;
b. removing the solution from flash drum 1 and contacting the solution with a second flocculant at a predetermined flow rate and passing the solution to flash drum 2 followed by re-incubating the solution for a predetermined duration and at a predetermined temperature along with continuous or intermittent skimming; and
c. removing the solution from flash drum 2 and contacting the solution with single or multiple dosages of chemicals including but not limiting to precipitants such as sodium hydroxide and sodium carbonate, and passing the solution to settling tank and allowing the solution to settle for a predetermined duration and at a predetermined temperature, for precipitation/sedimentation and separation of the colloidal suspension.

In a preferred exemplary embodiment, the system provides for separation of colloidal suspension from a solution of organic compound including but not limiting to hydrocarbon based solution such as rich MEG, in the following manner:
a. contacting the solution collected from the slug catcher with the first flocculant and pumping the solution to flash drum 1 at a flow rate of about 25M³/hr, and wherein the temperature of the solution is about 25° C.;
b. incubating the solution of step (a) in flash drum 1 for a time period ranging from about 10 minutes to about 15 minutes at a temperature ranging from about 80° C. to about 90° C., along with continuous or intermittent skimming;
c. removing the solution from flash drum 1 and contacting the solution with the second flocculant, followed by pumping the solution to flash drum 2;
d. re-incubating the solution in flash drum 2 for a time period of about 1 hour at a temperature ranging from about 80° C. to about 90° C., along with continuous or intermittent skimming; and
e. removing the solution from flash drum 2 and contacting the solution with single or multiple dosages of chemicals including but not limiting to precipitants such as sodium hydroxide and sodium carbonate, individually or in combination, and allowing the solution to settle for a predetermined duration and at a temperature ranging from about 80° C. to about 90° C., for precipitation and separation of the colloidal suspension.

In an embodiment, the first flocculant and the second flocculant are dosed at a flow rate ranging from about 1 lt/hr to about 2 lt/hr, respectively. In an alternate embodiment, the first flocculant and the second flocculant are dosed at a flow rate ranging from about about 0.0038%/hr to about 0.0072%/hr. In an exemplary embodiment, the FIGS. 4 to 18 illustrates the slurry/colloidal suspension settling capacity of the first flocculant and second flocculant at the flow rate ranging from about 1 lt/hr to about 2 lt/hr, respectively, wherein the individual concentration of the first flocculant and the second flocculant leads to efficient settling of the slurry/colloidal suspension at the bottom, thereby aiding in easy and efficient separation of slurry/colloidal suspension from the solution of organic compound including but not limiting to hydrocarbon based solution such as rich MEG to obtain clarified hydrocarbon based solution such as clarified MEG.

In a non-limiting embodiment, the first flocculant and/or the second flocculant are added at a concentration ranging from about 0.005% v/v to about 0.010% v/v, respectively in the system for separation of colloidal suspension from a solution of organic compound including but not limiting to hydrocarbon based solution such as rich MEG solution to obtain clarified hydrocarbon based solution such as clarified MEG. In a preferred embodiment the first flocculant and the second flocculant are independently added at a concentration ranging from about 0.007% to about 0.008%.

In yet another non-limiting embodiment, in the system of the present disclosure involving contacting the hydrocarbon based solution such as rich MEG to at least one flocculant followed by chemicals including but not limiting to precipitants such as sodium hydroxide and sodium carbonate, the sodium hydroxide is provided at a concentration ranging from about 40%w/v to about 50% w/v and the sodium carbonate is provided at a concentration ranging from about 10% w/v to about 40%w/v.

In an exemplary embodiment, the system of the present disclosure is further illustrated in FIG. 4. Although the figure exemplifies use of first flocculant, second flocculant and precipitants employed in the methods of the present disclosure, the figure is provided only for the purposes of illustration. The figure and the specifics mentioned therein with regards to the conditions and parameters should not be construed to limit the scope of the present disclosure. As is understood by a person skilled in the art, the system of the present disclosure is provided to perform the methods of the present disclosure, whereby the methods are performed by employing the flocculants and/or the chemicals such as precipitants in the concentration ranges provided herein and at conditions and parameters mentioned in the present disclosure and previous embodiments.

In an exemplary embodiment, the methods and system of the present disclosure for separation of colloidal suspension from a solution of organic compound including but not limiting to hydrocarbon based solution such as rich MEG, provides for about 65% to about 85% oil recovery from said solution.

In another exemplary embodiment, the methods and system of the present disclosure leads to about 40% separation of the slurry/colloidal suspension. Further, the methods and system of the present disclosure provides for recovery for about 100% recovery of clarified hydrocarbon based solution such as clarified MEG.

In a non-limiting embodiment, the methods and system for separation of colloidal suspension of the present disclosure provides for:
enhanced oil recovery ranging from about 65% to about 85% from the hydrocarbon based solution such as rich MEG;
faster and better slurry/colloidal suspension settling in the MEG settling tank, wherein about 95% to about 100% of the slurry/colloidal suspension settling is achieved, thereby leading to about 95% to about 100% separation of the colloidal suspension from the solution of organic compound;
reduction in choking due to the deposition of salts in pipelines of reclamation unit and pretreatment system;
increase in the performance of the reclamation unit and centrifugation operation post settling of impurities; and
About 95% to about 100% recovery of clarified hydrocarbon based solution such as clarified MEG from the hydrocarbon based solution such as rich MEG;

Additional embodiments and features of the present disclosure will be apparent to one of ordinary skill in art based upon description provided herein. The embodiments herein provide various features and advantageous details thereof in the description. Descriptions of well-known/conventional methods and techniques are omitted so as to not unnecessarily obscure the embodiments herein. The examples provided herein are intended merely to facilitate an understanding of ways in which the embodiments herein may be practiced and to further enable those of skill in the art to practice the embodiments herein. Accordingly, the following examples should not be construed as limiting the scope of the embodiments herein.

### EXAMPLES

### Example 1

### Demonstration of MEG Treatment Upon Addition of First Flocculant and Second Flocculant, Followed by Sodium Hydroxide and Sodium Carbonate

About 0.006% of first flocculant is added to rich MEG solution obtained from a slug catcher, and the solution is allowed to settle for about 10 minutes to about 15 minutes at a temperature of about 80° C. and the top layer comprising oil is skimmed. To the skimmed solution, about 0.006% of second flocculant solution is added and allowed to settle for a time period of about 1 hr at a temperature of about 80° C. to about 90° C. and the top layer comprising oil is skimmed. FIG. 1 illustrates the dosing of first and second flocculant. To the skimmed solution, about 50% of NaOH and about 25% of Na₂Co₃ is added and allowed to settle for a time period of about 10 minutes to about 20 minutes.

### Observation:

The rate of settling is very high and all the low solubility salts of divalent cations of iron, calcium, magnesium, barium and strontium are precipitated. About 65% to about 85% of oil is recovered. FIG. 3 illustrate percentage recovery of oil and FIG. 2 illustrates the percentage of the slurry.

### Example 2

### Demonstration of MEG Treatment Upon Addition of Only One Flocculant (Palm Oil and Palm Kernel Oil) followed by Sodium Hydroxide and Sodium Carbonate

The rich MEG solution from the slug catcher is heated to a temperature of about 85° C. for a duration of about 15 minutes, to this heated rich MEG solution, about 0.01% of second flocculant solution is added and allowed to settle for a time period of about 1 hr at a temperature of about 80° C. and the top layer comprising oil is skimmed. To the skimmed solution, about 0.8% of 50% NaOH solution and about 1.2% of 25% Na₂Co₃ solution is added. Thereafter the solution is allowed to settle for a predetermined duration.

### Observation:

The rate of settling is very high and all the low solubility salts of divalent cations of iron, calcium, magnesium, barium and strontium are precipitated. About 65% to about 85% of oil is recovered.

### Example 3

### Demonstration of MEG Treatment Upon Addition of First Flocculant, Followed by Sodium Hydroxide and Sodium Carbonate and Followed by Second Flocculant

The rich MEG solution from the slug catcher is heated to a temperature of about 90° C. for a duration of about 15 minutes, to this heated rich MEG solution, about 0.005% of first flocculant is added and the solution is allowed to settle for about 10 minutes to about 15 minutes at a temperature of about 90° C. and the top layer comprising oil is skimmed. To the skimmed solution, about 0.4% of 50% NaOH solution and about 0.6% of 25% Na₂CO3 is added, followed by stirring the solution for a predetermined duration. To this stirred solution about 0.005% second flocculant is added, followed by stirred the solution for a predetermined duration. Thereafter the solution is allowed to settle for a predetermined duration.

### Observation:

The rate of settling is very high and all the low solubility salts of divalent cations of iron, calcium, magnesium, barium and strontium are precipitated. About 65% to about 85% of oil is recovered.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments in this disclosure have been described in terms of preferred embodiments, those skilled in the art will recognize that the embodiments herein can be practiced with modification within the spirit and scope of the embodiments as described herein.

Throughout this specification, the word “comprise”, or variations such as “comprises” or “comprising” wherever used, will be understood to imply the inclusion of a stated element, integer or step, or group of elements, integers or steps, but not the exclusion of any other element, integer or step, or group of elements, integers or steps.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

While considerable emphasis has been placed herein on the particular features of this disclosure, it will be appreciated that various modifications can be made, and that many changes can be made in the preferred embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

1. A method for separation of colloidal suspension from solution of organic compound, said method comprising steps of:
a. contacting a solution of organic compound with at least one flocculant, followed by incubating the solution; and
b. contacting the solution of step (a) with at least one precipitant and allowing the solution to settle for separation of a colloidal suspension from the solution of organic compound.

2. The method as claimed in claim 1, wherein the step (a) is carried out by contacting the solution of organic compound with a first flocculant, and contacting the solution with a second flocculant, followed by incubating the solution.

3. The method as claimed in claim 2, wherein the solution of organic compound is heated to a temperature of about 80° C. to about 90° C. for a duration ranging from about 10 minutes to about 15 minutes prior to contacting the solution with the first flocculant and the second flocculant, independently.

4. The method as claimed in claim 1, wherein the step (a) is repeated at least once after contacting the solution with the precipitant.

5. The method as claimed in claim 1, wherein the flocculant comprises palm oil, palm kernel oil, water or any combination thereof; wherein the flocculant comprises palm oil at a concentration ranging from about 30% v/v to about 70% v/v; palm kernel oil at a concentration ranging from about 30% v/v to about 70%) v/v and water at a concentration ranging from about 10%>v/v to about 40%>v/v; or the flocculant comprises palm oil at a concentration ranging from about 40%>v/v to about 60%) v/v, palm kernel oil at a concentration ranging from about 30%>v/v to about 50% v/v and water at a concentration ranging from about 10%>v/v to about 40%) v/v; or the flocculant comprises palm oil at a concentration ranging from about 30%) v/v to about 70%>v/v and palm kernel oil at a concentration ranging from about 30%) v/v to about 70%>v/v; and wherein the flocculant is at a concentration ranging from about 0.005% v/v to about 0.01% v/v.

6. The method as claimed in claims 2, wherein the first flocculant comprises palm oil at a concentration ranging from about 40% v/v to about 60% v/v, palm kernel oil at a concentration ranging from about 30% v/v to about 50% v/v and water at a concentration ranging from about 10% v/v to about 40% v/v; the second flocculant comprises palm oil at a concentration ranging from about 30% v/v to about 70% v/v and palm kernel oil at a concentration ranging from about 30% v/v to about 70% v/v; and wherein the first flocculant and the second flocculant are independently at a concentration ranging from about 0.005%) v/v to about 0.01% v/v.

7. The method as claimed in claim 1, wherein the solution of organic compound is rich monoethylene glycol.

8. The method as claimed in claim 1, wherein the precipitant comprises sodium hydroxide, sodium carbonate, or a combination thereof and wherein the sodium hydroxide is at a concentration ranging from about 40% w/v to about 50%) w/v and sodium carbonate is at a concentration ranging from about 10%_w/v to about 40%_w/v.

9. The method as claimed in claim 1, wherein the incubation of the step (a) is carried out at a temperature ranging ranging from about 80° C. to about 90° C. for a duration ranging from about 10 minutes to about 15 minutes; and wherein the settling of the step (b) occurs at a temperature ranging from about 80° C. to about 90° C.

10. The method as claimed in claim 1, wherein the method comprises skimming of a hydrocarbon component comprising petroleum product, oil, natural gas, or any combination thereof, after addition of at least one flocculant to the solution of organic compound.

11. The method as claimed in claim 1, wherein the colloidal suspension comprises water, corrosion products, salt with moderate to low solubility, unwanted impurities, or any combination thereof, and wherein the moderate to low solubility salt comprises a divalent cation of metal comprising iron, calcium, magnesium, barium, strontium, or any combination thereof.

12. The method as claimed in claim 1, wherein recovery of clarified monoethylene glycol is ranging from about 95% to about 100%; and wherein recovery of oil is ranging from about 65% to about 85%.

13. The method as claimed in claim 1, wherein the separation of the colloidal suspension is ranging from about 95% to about 100%.

14. A system for separation of colloidal suspension from a solution of organic compound, the system comprising:
a. a first flash drum adapted to receive a mixture of a solution of organic compound and a first flocculant, and is configured to incubate the mixture;
b. a second flash drum fluidly contacted to the first flash drum, the second flash drum is adapted to receive the solution of organic compound from the first flash drum with a second flocculant, and is configured to incubate a mixture of the solution of organic compound and the second flocculant; and
c. a settling tank fluidly contacted to the second flash drum, the settling tank is adapted to receive the solution of organic compound from the second flash drum with a precipitant for the separation of a colloidal suspension from the solution of organic compound.

15. The system as claimed in claim 14, comprising at least one heating unit connected to at least one of the first flash drum and the second flash drum, wherein the heating unit supplies heat to at least one of the first flash drum and the second flash drum.

16. The system as claimed in claim 14, wherein inlets of the first flash drum, the second flash drum and the settling tank comprise at least one provision to receive the first flocculant and the second flocculant and the precipitant.

17. The system as claimed in claim 14, wherein the first flocculant and the second flocculant are added to the system at a flow rate ranging from about 1_lt/hr to about 2_lt/hr; wherein the first flocculant comprises palm oil at a concentration ranging from about 40% v/v to about 60% v/v, palm kernel oil at a concentration ranging from about 30%_v/v to about 50% v/v and water at a concentration ranging from about 10% v/v to about 40% v/v; the second flocculant comprises palm oil at a concentration ranging from about 30% v/v to about 70%) v/v and palm kernel oil at a concentration ranging from about 30%>v/v to about 70% v/v; and wherein the first flocculant and the second flocculant are at a concentration ranging from about 0.05% v/v to about 0.01% v/v, independently.

18. The system as claimed in claim 14, wherein the precipitant comprises sodium hydroxide, sodium carbonate or a combination thereof and wherein the sodium hydroxide is at a concentration ranging from about 40% w/v to about 50% w/v and sodium carbonate is at a concentration ranging from about 10%_w/v to about 40% w/v.

19. The system as claimed in claim 14, wherein the incubation of the step (a) is carried out at a temperature ranging from about 80° C. to about 90° C. for a duration ranging from about 10 minutes to about 15 minutes; the incubation of the step (b) is carried out at a temperature ranging from about 80° C. to about 90° C. for a duration of about 1 hour; and the settling of the step (c) occurs at a temperature ranging from about 80° C. to about 90° C.

20. The system as claimed in claim 19, wherein during the incubation, skimming of a hydrocarbon component is carried out, wherein the hydrocarbon component comprises petroleum product, oil, natural gas, or any combination thereof.

21. The system as claimed in claim 14, wherein the solution of organic compound is monoethylene glycol; and wherein the colloidal suspension comprises water, corrosion products, salt with moderate to low solubility, unwanted impurities, or any combination thereof, and wherein the moderate to low solubility salt comprises a divalent cation of metal comprising iron, calcium, magnesium, barium, strontium, or any combination thereof.

22. The system as claimed in claim 14, wherein recovery of clarified monoethylene glycol is ranging from about 95% to about 100%; wherein recovery of oil is ranging from about 65% to about 85%; and wherein the separation of the colloidal suspension is ranging from about 95% to about 100%.
